(19) United States
(12) Patent Application Publication
(10) Pub. No.: US 2018/0000540 A1
(43) Pub. Date: Jan. 4, 2018

(54) AN IMPROVED CATHETER AND METHOD OF MANUFACTURE THEREOF

(71) Applicant: CathRx Ltd, Homebush Bay, NSW (AU)

(72) Inventors: David Ogle, Cowan (AU); Roman Greifeneder, Bexley (AU)

(73) Assignee: CathRx Ltd, Homebush Bay, NSW (AU)

(21) Appl. No.: 15/537,303

(22) PCT Filed: Dec. 3, 2015
(86) PCT No.: PCT/AU2015/050763

§ 371 Date: Jun. 16, 2017

(30) Foreign Application Priority Data
Dec. 17, 2014 (AU) 2014905122
Mar. 19, 2015 (AU) 2015901002

(51) Int. Cl. A61B 18/14; A61B 5/042; A61B 5/00; A61B 18/00

(52) U.S. Cl. CPC A61B 18/1492; A61B 5/6857; A61B 5/0422; A61B 2018/00577; A61B 2018/00011; A61B 2018/1435; A61B 2218/002; A61B 2018/00214

(57) ABSTRACT

A sheath adapted for use with a catheter is disclosed comprising an electrical lead having a proximal end and a distal end and a lumen extending from the proximal end to the distal end, the electrical lead including a tubular member of non-conductive material. At least a first set of electrical conductors and a second set of electrical conductors extend from the proximal end to the distal end laid on the non-conductive tubular member, and an outer layer of non-conductive material is applied over the electrical conductors to cover the conductors. One or more electrodes are disposed on a distal portion of the sheath. Each electrode is in electrical communication with at least one of the electrical conductors through the outer layer. The first set of electrical conductors is helically wrapped around the lumen and the second set of electrical conductors is helically wrapped around the first set of electrical conductors.

### CROSS-REFERENCE TO RELATED APPLICATIONS

This application is a national phase entry under 35 U.S.C. §371 of International Patent Application PCT/AU2015/050763, filed Dec. 3, 2015, designating the United States of America and published in English as International Patent Publication WO 2016/094938 A1 on Jun. 23, 2016, which claims the benefit under Article 8 of the Patent Cooperation Treaty to Australian Patent Application Serial No. 2015901002, filed Mar. 19, 2015, and Australian Patent Application Serial No. 2014905122, filed Dec. 17, 2014.

### TECHNICAL FIELD

This application relates to a catheter, system, or method adapted to allow for the inclusion of multiple sets of wires within the body of the catheter.

### BACKGROUND

Electrophysiology catheters are commonly used in medical practice to examine and treat the heart. They may be inserted into the cardiovascular system of a patient through small punctures in the skin. They may then extend through a vein into the heart where they sense the electrical activity of the heart. Some of the electrophysiology catheters may be able to treat the heart by ablating the appropriate areas of the heart in case of certain types of aberrant electrical activity.

Several different electrode designs have been developed for treating heart arrhythmias such as atrial flutter and atrial fibrillation. Previous catheters have included limited amounts of electrodes to achieve the actual ablation of the required tissue. Typically, previous attempts at diagnostic catheters have included fewer than twenty (20) electrodes mounted or positioned on the distal end of the catheter. This electrode limitation is restricted by the packaging and encapsulation of the wires that feed the respective electrode with electricity and power. Poor space management of the wires typically leads to fewer electrodes capable of being mounted on the catheter sheath.

Additionally, there has been a long-felt need for catheters that enable the carriage of more than the traditional limited number of wire sets within the catheter. Previous examples have been typically limited to a single set of wires positioned around the outside of the catheter lumen, wherein the single set of wires has been encapsulated within the catheter sheath. This type of configuration typically limits the maximum number of wires within the catheter body to twenty (20) or less.

Any discussion of the prior art throughout the specification should in no way be considered as an admission that such prior art is widely known or forms part of common general knowledge in the field.

### BRIEF SUMMARY

### Problems to be Solved

It may be an aim or objective of the present disclosure to provide for a system or device that may allow for the inclusion of more than twenty (20) wires within the body of the catheter.

It may also be an aim or objective of the present disclosure to provide an improved distal end for a diagnostic catheter.

It is an object of this disclosure to overcome or ameliorate at least one of the disadvantages of the prior art, or to provide a useful alternative.

### Means for Solving the Problem

A first aspect of the this disclosure relates to a sheath adapted for use with a catheter, wherein the sheath comprises: an electrical lead having a proximal end and a distal end and a lumen extending from the proximal end to the distal end, the electrical lead including a tubular member of non-conductive material, at least a first set and a second set of electrical conductors extending from the proximal end to the distal end laid on the non-conductive tubular member, and an outer layer of non-conductive material applied over the electrical conductors to cover the conductors; one or more electrodes mounted on a distal portion of the sheath and wherein each electrode may be in electrical communication with at least one of the plurality of electrical conductors through the outer layer; and wherein the first set of electrical conductors may be helically wrapped around the lumen; and the second set of electrical conductors may be helically wrapped around the first set of electrical leads.

Preferably, the first and second sets of electrical conductors are separated from each other by a second non-conductive layer. The preferred catheter further comprises a third set of electrical conductors having a proximal end and a distal end and a lumen extending from the proximal end to the distal end, the electrical leads each including a tubular member of non-conductive material, a plurality of electrical conductors extending from the proximal end to the distal end laid on the non-conductive tubular member, and an outer layer of non-conductive material applied over the electrical conductors to cover the conductors.

The preferred sheath may include a third set of electrical conductors, which may be helically wrapped around the second set of electrical conductors. Preferably, the third and second sets of electrical conductors are separated from each other by a third non-conductive layer.

The distal portion of the sheath may include sixty (60) or more electrodes. The preferred diameter of the sheath may be between 0.33 mm and 2.33 mm. The preferred catheter may be an ablation catheter.

A second aspect of the disclosure relates to a sheath adapted for use with a catheter, wherein the sheath comprises: an electrical lead having a proximal end and a distal end and a lumen extending from the proximal end to the distal end, the electrical leads each including a tubular member of non-conductive material, at least a first set of electrical conductors extending from the proximal end to the distal end laid on the non-conductive tubular member, and an outer layer of non-conductive material applied over the electrical conductors to cover the conductors; one or more electrodes on or near a distal portion of the sheath and wherein each electrode may be in electrical communication with at least one of the electrical conductors through the outer layer; and wherein the distal portion may be selectively transformed by a user in a distorted configuration, wherein the distorted configuration includes a helix shape with a reduced diameter at both ends of the distal portion.

Preferably, the first set of electrical conductors may be adapted to be wound around the lumen in a helix in a first direction. The sheath further comprises a second set of electrical conductors spaced apart from the first set of electrical conductors and separated by a non-conductive layer of material.

The preferred second set of electrical conductors may be adapted to be wound around the lumen in a helix in a second direction, which may be opposed to the first direction.

A third aspect of this disclosure relates to a sheath adapted for use with a catheter, wherein the sheath may comprise at least one electrical lead having a proximal end and a distal end and a lumen extending from the proximal end to the distal end, the at least one electrical lead comprising a tubular member of non-conductive material, at least a first set of electrical conductors may extend from the proximal end to the distal end laid on the non-conductive tubular member, and an outer layer of non-conductive material applied over the electrical conductors to cover the conductors; one or more electrodes on or near a distal portion of the sheath and wherein each electrode may be in electrical communication with at least one of the electrical conductors through the outer layer; and wherein the distal portion of the sheath may be a helix shape, in which the helix shape may comprise a first loop structure and a second loop structure such that the second loop structure may be spaced proximally relative to the first loop structure.

Preferably the first loop structure may comprise at least one sensing electrode.

Preferably, the second loop structure may comprise at least one ablation electrode. The second loop structure may comprise a plurality of fluid conduits to expel fluid.

Preferably, the first set of electrical conductors may be adapted to be wound around the lumen in a helix in a first direction. A second set of electrical conductors may be wound around the first set of electrical conductors in an opposing direction.

Preferably, the second set of electrical conductors may be spaced apart from the first set of electrical conductors by a non-conductive layer.

In the context of this disclosure, the words “comprise,” “comprising,” and the like, are to be construed in their inclusive, as opposed to their exclusive, sense, that is, in the sense of “including, but not limited to.”

The disclosure is to be interpreted with reference to the at least one of the technical problems described or affiliated with the background art. This disclosure aims to solve or ameliorate at least one of the technical problems, which may result in one or more advantageous effects as defined by this specification and described in detail with reference to the preferred embodiments of this disclosure.

### BRIEF DESCRIPTION OF THE DRAWINGS

FIG. 1 depicts a front perspective view of the cutaway aspects of a first preferred embodiment of this disclosure;

FIG. 2 depicts a side perspective view of the distal tip of a preferred catheter forming part of the first preferred embodiment of this disclosure;

FIG. 3 depicts the internal shape-forming member and steering mechanism;

FIG. 4 depicts a side perspective view of an embodiment of a preferred catheter of this disclosure.

FIG. 5 depicts a perspective view of an embodiment of a distal tip of a catheter of this disclosure;

FIG. 6 depicts another embodiment of a distal tip of a catheter of the disclosure with a sensing portion and an ablation portion;

FIG. 7A depicts an embodiment of a distal tip of a catheter of the disclosure with radial irrigation;

FIG. 7B depicts a similar embodiment to the structure as illustrated in FIG. 7A;

FIG. 8A illustrates an enlarged view of an embodiment of a portion of a distal end of a catheter with a printed electrode;

FIG. 8B illustrates an enlarged view of another embodiment of a portion of a distal end of a catheter with a printed electrode; and

FIG. 8C depicts a front perspective view of the cutaway aspects of another preferred embodiment of this disclosure with a printed electrode.

### DETAILED DESCRIPTION

Preferred embodiments of the disclosure will now be described with reference to the accompanying drawings and non-limiting examples.

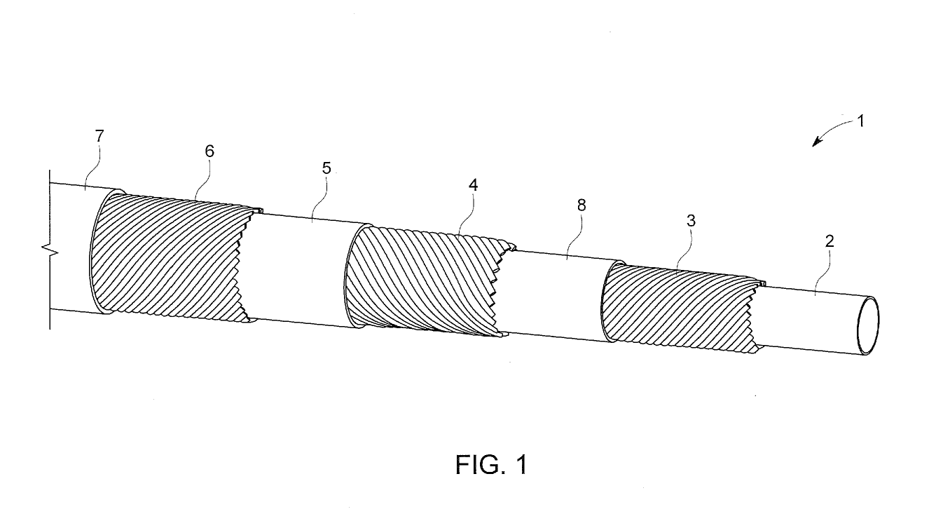

A first preferred embodiment of this disclosure is depicted in FIGS. 1 through 3. According to the first preferred embodiment, a catheter is provided wherein the catheter is preferably adapted for use as a diagnostic catheter but other uses are possible. The portion of the catheter shown in FIGS. 1 through 3 is the portion that relates to the sheath.

The catheter sheath 1 includes an elongated resilient but flexible body 21 having a distal end 23 and a proximal end (not shown). The proximal end of the sheath is adapted to be mounted or attached to a catheter handle (not shown), wherein the catheter handle may be manipulated by a clinician to allow for the maneuvering of the catheter during in situ use. Preferably, the sheath is constructed of biocompatible materials with respect to blood- or tissue-contacting regions or areas. The wires included within the constructions may be of any electrical conductive material, however, Litz wire or copper wire is preferred. The sheath 1 preferably encapsulates the wires in non-conductive, flexible but resilient biocompatible polymer, which may include PEBAX®, polyurethane or silicone polymers.

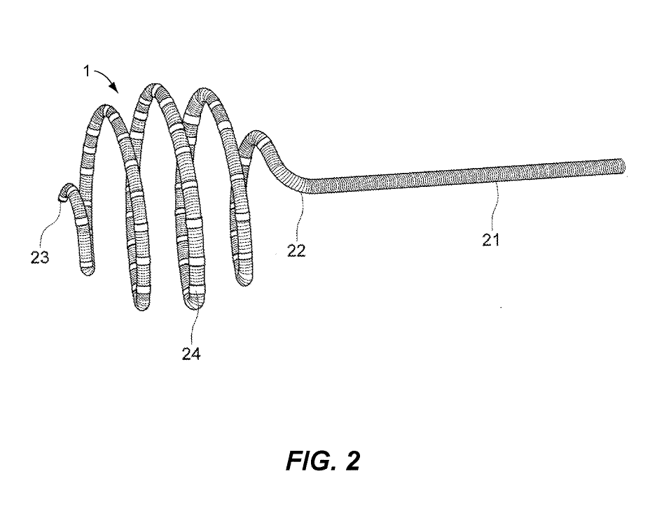

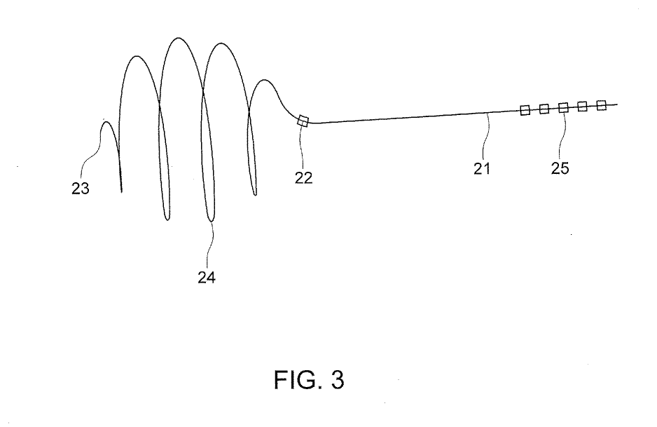

The distal end 23 is preferably adapted so that the tip may be inserted into a chosen artery of a patient and fed along the interior of the artery to a point proximal to an atria of the heart. The catheter handle may be manipulated by the clinician, which may cause the distal end to distort from a standard linear configuration (not shown) to the distorted configuration shown in FIGS. 2 and 3.

In the distorted configuration, the body of the catheter proximal to the distal end is rotated into a helical structure, wherein the helical structure includes a reducing radius at either end of the helical structure. The helical structure is depicted in FIGS. 2 and 3 wherein the body of the catheter is rotated preferably five times around the longitudinal axis of the body. The helical structure may also be described as forming a general ball or spherical shape as shown between the distal end 23 and point 22.

FIGS. 2 and 3 depict a sheath 1 for use with a cardiac ablation catheter wherein the preferred diameter of the sheath is 3 F. In this specification, “F” or “Fr” means French Scale or French Gauge and is a standardized term to measure the size or diameter of catheters and needles. In this specification, 1 F equals 0.33 mm.

The following details possible embodiment sizes that are able to be manufactured and created pursuant to this disclosure.

| | | No. of wiring | Preferred amount |
|---|---|---|---|
| Diameter (F) | Diameter (mm) | layers | of electrodes |
| 2 F | 0.67 mm | 2 | 20 |
| 3 F | 1 mm | 2 | 40 |
| 6 F | 2 mm | 3 | 80 |
| 7 F | 2.33 mm | 4 | 120 |

It will be appreciated that other French diameters may also be used, for example, 4.5 Fr (1.5 mm) which is a diameter in between 4 Fr and 5 Fr. Additionally, diameters larger than 7 Fr may also be used, such as 9 Fr (3 mm) or 12 Fr (4 mm).

FIGS. 2 and 3 depict a sheath with about seventy (70) electrodes 24 mounted or positioned near to the distal end 23 of the sheath 1. The tip of the distal end 23 is preferably rounded in profile to prevent damage to the patient during implantation or insertion of the device. The rounded tip may also additionally prevent accidental puncturing of the heart wall or cardiac tissue during implantation.

The helical structure is preferably used to perform cardiac ablation surgery with better accuracy than previous catheters. The relatively high numbers of electrodes mounted or positioned near the distal end 23 may provide for better mapping of the ECG signals along the heart wall.

Preferably, the electrodes 24 are attached to the sheath 1 by either one or more of the following methods: compression, clamping, heating or gluing (adhesive).

The linear portion of the sheath 1 is depicted as portion 21, wherein this portion 21 retains its linear structure during use. Point 22 is where the linear portion 21 meets and joins with the helical structure of the distal end 23. It will be appreciated that the linear structure may flex or bend along the path of insertion to arrive at the target location. Preferably, the linear portion 21 resists plastic deformation and returns to a predefined shape.

The shape-forming member in FIG. 3 is preferably made of or constructed of Nitinol. The shape-forming member may be optionally transformed or otherwise manipulated into a distorted shape. The distorted shape may be, for example, a helix shape with uniform or differing sized helix loops.

Portion 21 in FIG. 3 is the deflection region of the shape-forming member and includes a series of brackets or cutouts mounted on the sheath 1 close to or near to the proximal end of the sheath. These cutouts 25 may be used to soften the stylet to increase bending or general flexing of the catheter during insertion into a patient.

Alternatively, cutout sections may be selectively placed or positioned along the length of the sheath 1 to increase bending or flexing in a localized region. The overall stiffness of the sheath may be adjusted to suit the varying needs of specific applications or insertion locations and to allow for correct anatomical placement of the catheter.

Preferably, point 22 may be a point of termination between the helical structure and linear portion of the sheath 1. It may be necessary to fabricate or manufacture the helical structure and linear portion 21 separately and then at a later stage join the respective structures together at point 22. In FIG. 3, a joiner element is shown at point 22 to attach the distal portion of the sheath to the lower or remainder end of the sheath.

FIG. 1 depicts a cross-sectional view of the sheath 1, wherein the various layers of the sheath have been peeled back to expose the interior and the view is magnified. Typically, FIG. 1 depicts the 3 F diameter sheath. The sheath 1 includes a first layer of insulative material, lumen 2 herein, for insertion of the shape-forming member.

Mounted or positioned around the tube of the lumen is a first set of wires 3. The first set of wires 3 are wrapped in a tight helical pattern or fashion around the tube in a counterclockwise direction. The preferred wires within each set of wires are tightly packed and abut against each other. Helical winding of the wires allows for greater flexibility of the sheath 1 and a reduced risk of wire breakage from repeated bending.

Preferably, the lumen 2 of the first preferred embodiment may have a diameter of between 0.5 mm to 1 mm. The lumen size or diameter may be increased to selectively increase the flexibility of the sheath 1. In some embodiments, the selectively deflectable regions, such as between point 22 and the distal end 23, may have a diameter of 1 mm, while regions not requiring as much flexibility or non-deflectable regions such as between point 22 and the proximal end may have a diameter of 0.5 mm.

Mounted on top of the first set of wires 3 is a second layer 8 of insulative material. Preferably, the second layer 8 is positioned between the first set of wires 3 and the second set of wires 4. The second set of wires 4 is preferably wrapped helically around the outer surface of the second layer 8 of insulative material in a direction opposite the winding direction of the first set of wires 3. In this embodiment, the winding of the second set of wires 4 is in a clockwise direction.

A third layer 5 of insulative material is placed, molded or positioned over the second set of wires 4. The third layer 5 is adapted to encapsulate the second set of wires 4 to prevent fluid ingress from the patient's body when in use. The layers of insulative material also may serve to limit EMF interference of the sets of wires being in close proximity. In alternative embodiments, not shown in the figures, EMF braided shielding may also be inserted into one or more of the layers of insulative material to further limit EMF interference from neighboring wires and the external environment.

In this embodiment, a third set of wires 6 has been mounted or positioned above the third layer 5 of insulative material. The third set of wires 6 is additionally encapsulated within a further fourth layer 7 of insulative material. Preferably, the layers of insulative material are constructed of similar material. The third set of wires 6 is wound in a helical pattern in an opposite direction to the previous (beneath) set of wires, which is the second set of wires 4 in this example. The third set of wires 6 is wound in this embodiment in a counterclockwise direction.

Preferably, various embodiments of this disclosure may include varying amounts of wiring sets. It is preferred that the winding of the wiring sets be in opposite directions. The counter winding of the various wiring sets allows for the wires to be in a configuration that is similar to a braid or may be similar to a weave or knit. The configuration of opposed winding directions may allow for multiple sets of wiring to be layered upon each other while still allowing for overall flexibility of the sheath without damage or breakage.

The dimensions of the alternative wiring possibilities are described in the aforementioned table, which provides examples of possible configurations that may be achieved by the disclosure.

Preferably, the sheath may include a generally higher number of electrodes when compared to the prior art in this field. The multiple layers of wiring allow for more than twenty (20) electrodes 24 to be mounted on the outer surface or layer 7 of the sheath 1. In the first preferred embodiment, shown in FIGS. 1 through 3, the sheath 1 includes seventy (70) electrodes, wherein each electrode is attached to a separate wire within the sheath. The density of wiring and its associated sets allows for a multitude of electrodes to be used, which may provide better accuracy, better results and better resolution of ablation.

Preferably, wires for the electrodes are relatively equally divided between the wiring sets. Typically, the remainder is resolved by additional wires in the outermost wiring sets.

In further embodiments of this disclosure, a 6 F diameter sheath could be adapted to carry three layers of wiring sets and thereby allow a maximum of 200 electrodes to be mounted on the distal portion. Although, the more preferred amount may be 80 electrodes in this configuration.

A 7 F diameter sheath could be adapted to four layers of wiring sets and thereby allow for a maximum of 300 electrodes to be mounted on the distal portion. Although, the more preferred amount may be 120 electrodes in this configuration.

The most preferred diameters of the sheath are between 3 F and 9 F, however, up to 12 F may be used but the flexibility of the sheath may be reduced. Increasing the diameter of the sheath, however, may also improve the wear resistance of the catheter.

In alternative embodiments, fourth and fifth wiring sets may be added to the embodied sheath to adapt the sheath to carry more wires in total, and thereby carry more electrodes on the distal portion of the sheath.

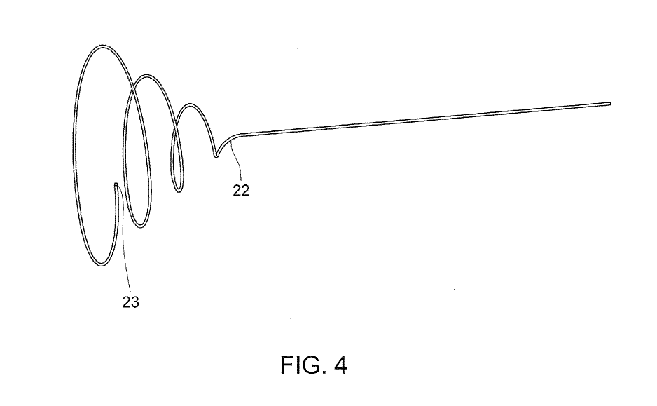

Another embodiment is shown in FIG. 4, wherein the helix structure between distal end 23 and point 22 has been replaced with an alternative structure. In this embodiment, the helix structure forms a conical shape extending toward the distal end, wherein the maximum radius of the helix is located near the distal end 23 and the apex of the cone shape is directed to the point 22. This structure may aid in placing the catheter against the interior walls of the heart when in use.

Further embodiments (not shown), may include the helix structure being positioned between the distal end 23 and point 22, wherein the helix structure includes a constant radii (or French) between distal end 23 and point 22 to form a tube-like structure. This structure may also have an advantage in aiding in the placement of the catheter against the interior walls of the heart when in use.

In this specification, the term “distal portion” refers to the region of the sheath 1 between the distal end 23 and point 22. Further, “electrical conductors” may include wires within its meaning. Further, references to “insulative” mean the equivalent to “non-conductive.”

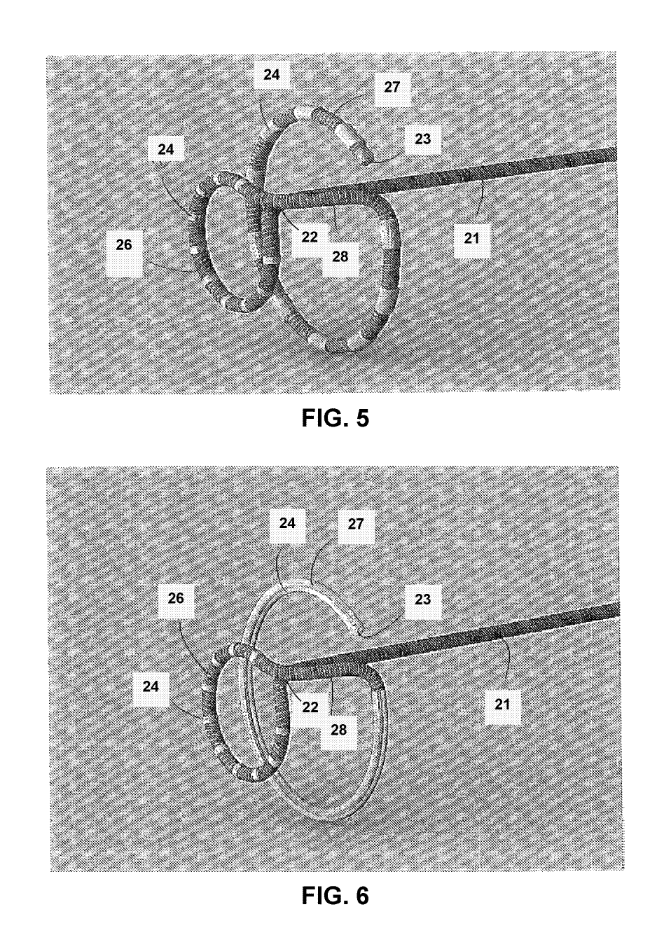

Yet another embodiment of the catheter sheath is illustrated in FIG. 5, wherein the helix structure between the distal end 23 and the point 22 has been replaced with an alternative structure. In this embodiment, the helix structure forms a first loop structure 26 near the point 22 and a second loop structure 27 proximally spaced from point 22 in the direction of the linear portion 21. At least one electrode may be disposed on at least one of the first and second loop structures 26 and 27. Preferably, a plurality of electrodes is disposed on at least one of the first and second loop structures 26, 27 and may be formed, for example, from a computer numerical control (CNC) machined from platinum or other biocompatible electrode material, such as gold, and is fixed to the sheath by swaging, adhesive or heating. The first loop structure 26 and the second loop structure 27 may be attached by at least one bridge member 28. The bridge member 28 spaces the first loop structure 26 and the second loop structure 27 at a desired distance or a predetermined distance. Alternatively, the first loop structure 26 and the second loop structure 27 may be exclusively attached to the linear portion 21.

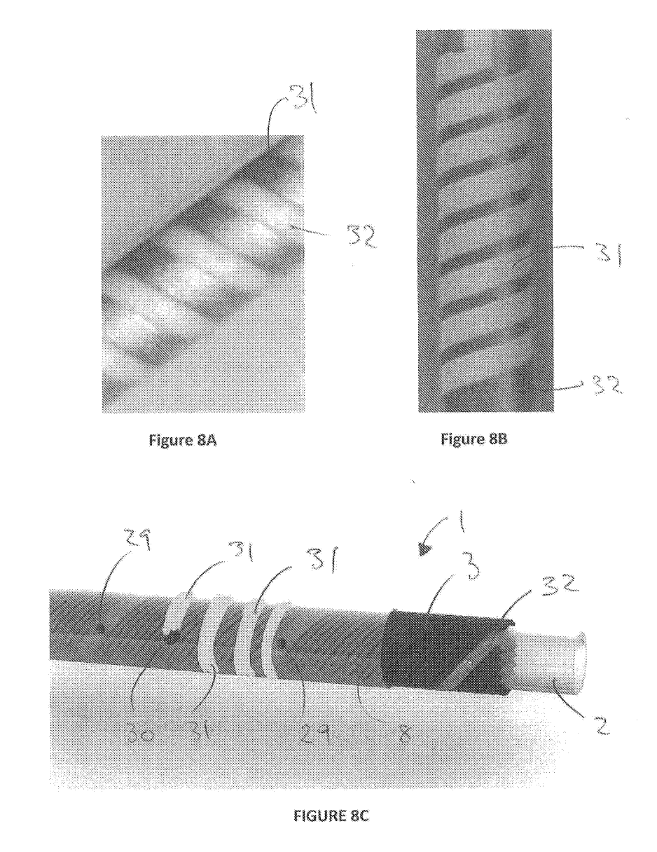

The first loop structure 26 may be a sensing loop 26 and the second loop structure 27 may be an ablation loop 27. The use of a double loop may reduce the skill and operation time required by an operator, as a double loop allows the operator to locate and burn lesions more efficiently than some known devices. Similar to the above embodiments, the helix structure may have a lumen with a first set of electrical conductors adapted to be wound around or embedded around the outer surface of the lumen in a first direction. Preferably, the winding of the wires is a helix at approximately 45 degrees relative to the longitudinal axis of the lumen. Other winding orientations may also be desirable, such as 30 degrees to 45 degrees, as changing the winding orientation may alter the stiffness or flexibility of the catheter. The sheath 1 (FIG. 1) may further comprise a second set of electrical conductors spaced apart from the first set of electrical conductors and separated by a non-conductive layer of material. More than a first set 3 and second set 4 of electrical conductors may be used and may optionally be spaced apart by a non-conductive layer 8. Each set of electrical conductors, after the first set 3 of electrical conductors, is preferably wound in an opposing direction to that of the previous set of electrical conductors. It will also be appreciated that the electrical conductors (or wires) may be spaced regularly, intermittently, or in a predetermined irregular configuration such that they impart a desired flexibility to the sheath 1 or other desirable property. The electrical conductors may have spacing (not shown) to allow for cuts to be formed between the electrical conductors. Optionally, if the electrical conductors are spaced, a non-conductive material 32 may be positioned between the electrical conductors to maintain a uniform spacing or to restrict the movement of the electrical conductors (see FIGS. 8A through 8C).

Turning to FIG. 6, there is illustrated another embodiment using the helix arrangement shown in FIG. 5. This embodiment depicts a second loop 27 with a single electrode. The electrode may also optionally extend through at least a portion of the lumen of the second loop 27, as opposed to being disposed on the outer surface. Extending the electrode through only a portion of the second loop 27 may reduce manufacturing costs. Alternatively, the second loop 27 may be formed from a conductive material, such as a biocompatible metal or a biocompatible conductive polymer and act as an electrode.

A guide wire or stylet (not shown) may be used to impart a preformed or desired shape, such as a loop or helix to the distal end of the catheter as illustrated in the figures. In at least another embodiment, an electrode, a core wire or a preformed catheter sheath may be adopted to impart a preformed or desired shape to the distal end of the catheter. Other shapes may also be imparted to at least one of the first loop structure 26 or the second loop structure 27. Optionally, the sheath 1 may comprise a guide wire as well as a preformed section to impart shape to the distal end 23 of the sheath 1.

It will be appreciated that the first loop structure 26 and second loop structure 27 can be disposed substantially concentrically to the linear portion 21 or can be disposed radially offset relative to the linear portion 21. In at least one embodiment, the first loop and second loop structures 26 and 27 can be axially or radially offset relative to each other or be disposed at different angles (not shown). This is advantageous as this can allow treatment of tortuous anatomy or undulating tissue within a patient during use.

In at least one embodiment, the at least one electrode may be flexible such that the helix structure can be maneuvered to a target location by a clinician or physician within tortuous anatomy, while minimizing the potential for damage of tissue or organs of a patient. Further, the size or shape of the loops may be altered by a manipulation means (not shown) to allow easier insertion along the path of insertion or provide a more effective operative structure. The size or shape of the first loop structure 26 and second loop structure 27 may be manipulated independently or in combination. Preferably, the diameter of the first loop structure 26 is less than that of the second loop structure 27 to provide an improved anatomical fit.

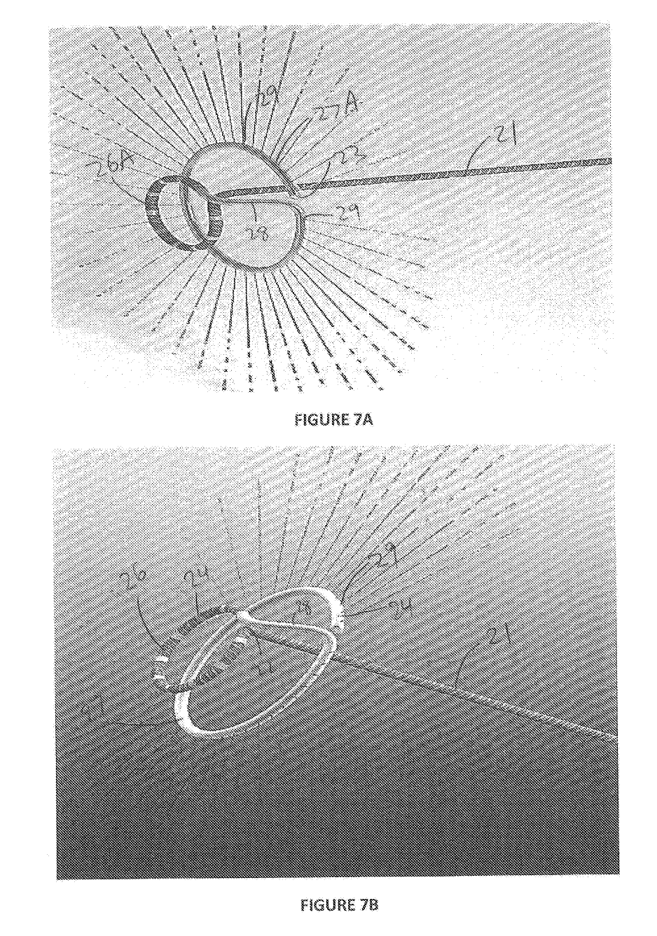

Referring now to FIGS. 7A and 7B, there is illustrated another embodiment of the disclosure. The helix structure between the distal end 23 and the point 22 has been replaced with an alternative helix structure similar to that shown in FIG. 5. The helix may comprise a first loop and a second loop that have been flattened to make a flat first loop 26A and a flat second loop 27A. The flat first loop 26A and flat second loop 27A may be a flat sensing loop 26A and a flat ablation loop 27A, respectively, wherein the flat ablation loop 27A comprises a number of irrigation apertures 29 through which irrigation fluid, such as a saline fluid or other fluid known in the art, may be expelled. The irrigation apertures 29 of the flat ablation loop 27A can be formed by known methods, such as laser cutting, and may be in a regularly spaced aperture 29 arrangement to evenly deliver fluid to a target area. Alternatively, the apertures 29 may be variable depending on the application of the catheter. Fluid may be selectively energized by the electrodes, which can be either disposed inside the lumen of the flat ablation loop 27A or externally disposed on the flat ablation loop 27A.

FIG. 7B differs from FIG. 7A in that the first and second loops are generally a tubular structure as opposed to a flat loop structure. Further, the first and second loop structures 26, 27 may be perpendicular to the linear portion 21 or may be angled relative to the linear portion 21 as illustrated in FIG. 7A. Preferably, the first loop structure 26 and the second loop structure 27 are substantially parallel to each other.

In at least one embodiment, the electrodes can be in a substantially planar arrangement such that significant height differences between the electrodes and the sheath are minimized or removed. This effectively removes or reduces the possibility of a blood clot forming within the vascular system or along the path of insertion that impedes blood flow.

In at least one embodiment, the first loop structure 26 and the second loop structure 27 are formed of a differing French (Fr) diameter. Preferably, the first loop structure, or distal loop, can be a 3 Fr diameter and the second loop structure 27, or proximal loop, can be a 4.5 Fr to 7 Fr diameter. It will be appreciated that other diameter sizes may be used or the first loop structure 26 may be larger than the second loop structure 27. Having differing loop sizes may provide a catheter that is more easily maneuvered along the path of insertion or may be used to increase or decrease the ablation zone while maintaining a desired sensing electrode configuration.

The first loop structure 26 is preferably formed by heat setting a Nitinol wire into a ring shape. The sheath of the helix may comprise swaged rings over a wound wire cable or a non-conductive layer. The wire cable may be in electrical communication with the swaged rings through an electrical conduit 30 (see FIG. 8C). The second loop structure 27 may be formed in a similar manner to that of the first loop structure 26 or fabricated separately and joined at a later time.

FIGS. 8A through 8C illustrate another embodiment of the disclosure in which the electrodes 24 are formed from a printed or flat conductor 31 fixed to the sheath 1. Printing an electrode 24 on the sheath 1 can result in a uniform arrangement, which can reduce the change of clots forming in the insertion path of the catheter and provides a more even ablation of lesions and reduces the possibility of gaps forming during ablation. Further, the use of printed electrodes 24 may also increase the tissue surface contact reducing the operation time. It will be appreciated that a uniform arrangement, such as a spaced electrode arrangement may be adopted, or an irregular arrangement (FIG. 8C) may be adopted for alternating spacing of electrodes 24 or for tailored specialized ablation procedures. A spaced electrode 24 arrangement may allow irrigation conduits 29 to be formed in the sheath 1 while avoiding cutting the electrodes 24 and maintaining integrity of the electrodes.

In a further embodiment, more than two loop structures (not shown) may be adopted. Each additional loop structure may comprise at least one electrode 24 or no electrodes and may be used to provide a more structurally stable catheter in the anatomy of a patient or may be placed intermediate the first loop structure and the second loop structure to allow altered spacing of the helix. Each loop may be connected by at least one bridge member 28 from a preceding loop structure or connected to the linear portion 21 (not shown). The bridge member 28 may be skewed or oriented at a different angle, or is otherwise not along the same axis as that of the linear portion 21. This may allow the first and second loop structures 26, 27 to be disposed parallel and/or generally concentric relative to one another. Optionally, the bridge member 28 may form part of the second loop structure 27 electrode.

Although the disclosure has been described with reference to specific examples, it will be appreciated by those skilled in the art that the disclosure may be embodied in many other forms, in keeping with the broad principles and the spirit of the disclosure described herein.

This application and the described preferred embodiments specifically include at least one feature that is industrially applicable.

1. A sheath adapted for use with a catheter, wherein the sheath comprises:
an electrical lead having a proximal end and a distal end and a lumen extending from the proximal end to the distal end, the electrical lead including a tubular member of non-conductive material, at least a first set of multiple electrical conductors and a second set of multiple electrical conductors extending from the proximal end to the distal end laid on the non-conductive tubular member, and an outer layer of non-conductive material applied over the multiple electrical conductors to cover the conductors;
one or more electrodes disposed on a distal portion of the outer layer and wherein each electrode is in electrical communication with at least one of a plurality of electrical conductors through the outer layer; and
wherein the first set of multiple electrical conductors is helically wrapped around the lumen; and the second set of multiple electrical conductors is helically wrapped around the first set of multiple electrical conductors.

2. The sheath of claim 1, wherein the first and second sets of multiple electrical conductors are separated from each other by a second non-conductive layer.

3. The sheath of claim 2, wherein the catheter further comprises a third set of multiple electrical conductors having a proximal end and a distal end.

4. The sheath of claim 3, wherein the third set of multiple electrical conductors is helically wrapped around the second set of multiple electrical conductors.

5. The sheath of claim 4, wherein the third and second sets of multiple electrical conductors are separated from each other by a third non-conductive layer.

6. The sheath of claim 1, wherein the distal portion includes sixty (60) or more electrodes.

7. The sheath of claim 6, wherein the diameter of the sheath is between 0.33 mm and 2.33 mm.

8. The sheath of claim 1, wherein the catheter is an ablation catheter.

9. A sheath adapted for use with a catheter, wherein the sheath comprises:
at least one electrical lead having a proximal end and a distal end and a lumen extending from the proximal end to the distal end, each of the at least one electrical leads including a tubular member of non-conductive material, at least a first set of electrical conductors extending from the proximal end to the distal end laid on the non-conductive tubular member, and an outer layer of non-conductive material applied over the electrical conductors to cover the conductors;
one or more electrodes on or near to a distal portion of the sheath and wherein each electrode is in electrical communication with at least one of the electrical conductors through the outer layer; and
a distal portion of the sheath may be selectively transformed by a user in a distorted configuration, wherein the distorted configuration includes a helix shape with a reduced diameter at both ends of the distal portion.

10. The catheter sheath of claim 9, wherein the first set of electrical conductors is adapted to be wound around the lumen in a helix in a first direction.

11. The catheter sheath of claim 10, wherein the sheath further comprises a second set of electrical conductors spaced apart from the first set of electrical conductors and separated by a non-conductive layer of material.

12. The catheter sheath of claim 11, wherein the second set of electrical conductors is adapted to be wound around the lumen in a helix in a second direction, which is opposed to the first direction.

13. A sheath adapted for use with a catheter, wherein the sheath comprises:
at least one electrical lead having a proximal end and a distal end and a lumen extending from the proximal end to the distal end, the at least one electrical lead comprising a tubular member of non-conductive material, at least a first set of electrical conductors extending from the proximal end to the distal end laid on the non-conductive tubular member, and an outer layer of non-conductive material applied over the electrical conductors to cover the conductors;
one or more electrodes on a distal portion of the outer layer and wherein each electrode is in electrical communication with at least one of the electrical conductors through the outer layer; and
a distal portion of the sheath being a helix shape, in which the helix shape comprises a first loop structure and a second loop structure such that the second loop structure is spaced proximally relative to the first loop structure.

14. The catheter sheath of claim 13, wherein the first loop structure comprises at least one sensing electrode.

15. The catheter sheath of claim 13, wherein the second loop structure comprises at least one ablation electrode.

16. The catheter sheath of claim 13, wherein the second loop structure comprises a plurality of fluid conduits to expel fluid.

17. The catheter sheath of claim 13, wherein the first set of electrical conductors is adapted to be wound around the lumen in a helix in a first direction.

18. The catheter sheath of claim 13, further comprising a second set of electrical conductors wound around the first set of electrical conductors in an opposing direction.

19. The catheter sheath of claim 18, wherein the second set of electrical conductors is spaced apart from the first set of electrical conductors by a non-conductive layer.

20. The catheter sheath of claim 13, wherein the first loop structure and the second loop structure are substantially parallel and joined by a bridge member.

21. The catheter sheath of claim 20, wherein the bridge member is not parallel to a longitudinal axis of the tubular member.
